(19) United States
(12) Patent Application Publication
(10) Pub. No.: US 2022/0409314 A1
(43) Pub. Date: Dec. 29, 2022

(54) MEDICAL ROBOTIC SYSTEMS, OPERATION METHODS AND APPLICATIONS OF SAME

(71) Applicant: AVRA Medical Robotics, Inc., Orlando, FL (US)

(72) Inventors: Alexandre Stephen Clug, Jupiter, FL (US); Mihir Lad, Suwanee, GA (US); Joseph Antonio Patullo, Orlando, FL (US); Farhan Taghizadeh, Phoenix, AZ (US); Onur Toker, Lakeland, FL (US); Mark Daniel Ellis, Lakeland, FL (US)

(21) Appl. No.: 17/903,202

(22) Filed: Sep. 6, 2022

Related U.S. Application Data
(63) Continuation-in-part of application No. 16/071,311, filed on Jul. 19, 2018.
(60) Provisional application No. 63/240,915, filed on Sep. 4, 2021.
(60) Provisional application No. 62/493,002, filed on Jun. 20, 2016.
(60) Provisional application No. 62/499,952, filed on Feb. 9, 2017.
(60) Provisional application No. 62/499,954, filed on Feb. 9, 2017.
(60) Provisional application No. 62/499,965, filed on Feb. 9, 2017.
(60) Provisional application No. 62/499,970, filed on Feb. 9, 2017.
(60) Provisional application No. 62/499,971, filed on Feb. 9, 2017.

(51) Int. Cl. A61B 34/37; A61M 37/00

(52) U.S. Cl. CPC A61B 34/37; A61M 37/0015; A61B 2034/305

(57) ABSTRACT

A robotic system for treating a patient includes a robotic arm with an end effector for treating the patient, wherein the robotic arm is configured to be movable in a space surrounding the patient, and the end effector is configured to be movable individually and co-movable with the robotic arm in said space; a sensing device for acquiring data associating with coordinates and images of the end effector and the patient; and a controller in communications with the robotic arm and the sensing device for controlling movements of the robotic arm with the end effector and treatments of the patient with the end effector based on the acquired data and a treatment plan.

### CROSS-REFERENCE TO RELATED PATENT APPLICATIONS

This application claims priority to and the benefit of provisional patent application Ser. No. 63/240,915 filed Sep. 4, 2021, which is incorporated herein in its entirety by reference.

This application is also a continuation in part application of patent application Ser. No. 16/071,311 filed Jul. 19, 2018, which is a national stage entry of PCT patent application No. PCT/US2017/038398 filed Jun. 20, 2017, which itself claims priority to and the benefit of provisional patent application Ser. No. 62/493,002 filed Jun. 20, 2016; provisional patent application Ser. No. 62/499,952 Feb. 9, 2017, provisional patent application Ser. No. 62/499,954 Feb. 9, 2017, provisional patent application Ser. No. 62/499,965 Feb. 9, 2017, provisional patent application Ser. No. 62/499,970 Feb. 9, 2017, and provisional patent application Ser. No. 62/499,971 filed Feb. 9, 2017, which are incorporated herein in their entireties by reference.

### FIELD OF THE INVENTION

The invention relates generally to the field of medical robotics, and more particularly to medical robotic systems, operation methods and applications of the same.

### BACKGROUND OF THE INVENTION

The background description provided herein is for the purpose of generally presenting the context of the present invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

Robot arms have been used in some surgical medical applications, but the robotic arm is generally controlled by a live human being that causes the robotic arm to move as directed in real time by the human operator. This means that a doctor generally must be present for the entire operation and is essentially operating remotely through the robotic arm rather than the robotic system.

It is possible to program a robotic arm to support a surgical or medical instrument and to move that instrument in a predetermined pattern. A consideration may arise, however, in that, if the patient moves during the procedure, a self-controlled robotic system performing medical, therapeutic or prophylactic procedures may lose track of the location of the patient or of the precise part of the patient where the robot is to operate.

The patient can of course be completely immobilized to prevent his or her movement, but that is normally very uncomfortable for a patient, especially in a medical procedure that takes more than a few minutes.

Therefore, a heretofore unaddressed need exists in the art to address the aforementioned deficiencies and inadequacies.

### SUMMARY OF THE INVENTION

It is therefore the object of this invention to provide a robotic system that is capable of performing operations on a patient without substantial amounts of human control and where the patient moves.

In one aspect, the invention relates to a robotic system for treating a patient. The robotic system comprises a robotic arm with an end effector for treating the patient, wherein the robotic arm is configured to be movable in a space surrounding the patient, and the end effector is configured to be movable individually and co-movable with the robotic arm in said space; a sensing device for acquiring data associating with coordinates and images of the end effector and the patient; and a controller in communications with the robotic arm and the sensing device for controlling movements of the robotic arm with the end effector and treatments of the patient with the end effector based on the acquired data and a treatment plan.

In one embodiment, the end effector is supported on a load sensor on the end of the robotic arm so that the end effector is movable by the robotic arm to essentially any location in said space.

In one embodiment, the load sensor is a three-axis load sensor for sensing the forces being applied to the end effector in three orthogonal axes, x, y and z, with the z-axis being the lengthwise axis of the end effector and the x- and y-axes being lateral dimensions perpendicular to that vertical dimension and each other.

In one embodiment, the end effector comprises an operative portion that acts on the patient directly, a tool control circuit for controlling actions of the end effector, and a housing containing the electronic circuitry, wherein a proximal end of a housing is supported on the load sensor, and a distal end of the housing supports the operative portion.

In one embodiment, the end effector is a surgical instrument or a medical instrument.

In one embodiment, the end effector is a scalpel, scissors, an electrocauterizer, a gas plasma treatment tool, and/or a microneedle treatment tool.

In one embodiment, the microneedle treatment tool comprises an array of microneedles configured such that each microneedle is selectively activatable to extend into or retract from the skin of the patient independently.

In one embodiment, the microneedle treatment tool is further configured to apply radio frequency (RF) waveforms, heat, light, and/or drug through the array of microneedles to the skin of the patient for therapy.

In one embodiment, the array of microneedles are supported on a structure in the end effector that selectively extends them out through a planar front face of the end effector and into the skin of the patient, or retracts them back behind the planar front face.

In one embodiment, a force applied to each microneedles to enter the skin of the patient is selected at varied levels.

In one embodiment, the sensing device comprises a first sensing unit and a second sensing unit, wherein the first sensing unit is disposed in said space at a stationary location vertically above the patient and directed at the patient, and the second sensing unit is attached on the end effector, such that during the treatment, the second sensing unit moves with the end effector, while the first sensing remains stationary on its support over the patient.

In one embodiment, each of the first and second sensor units comprises a LiDAR sensor and at least one camera, wherein the LiDAR sensor is configured to determine distances of the LiDAR sensor to surfaces of objects in its field of view, and the at least one camera is configured to acquire stereoscopic images of the objects in its field of view.

In one embodiment, the acquired data by each sensing unit comprises an array of range data, and video data for a field of pixels, wherein the range data for each pixel is an optically derived LiDAR distance value of the distance from the LiDAR sensor to the nearest object met by a ray extending through that pixel from the LiDAR sensor.

In one embodiment, the sensing device comprises a LiDAR sensor located in a center of the bed above the patient; and a first stereoscopic camera and a second stereoscopic camera symmetrically located a distance to the left and right of the LiDAR sensor, respectively, wherein the LiDAR sensor and the first and second stereoscopic cameras are attached on a stationary support.

In one embodiment, the controller is in wired or wireless communications with the robotic arm and the sensing device.

In one embodiment, the controller is configured to receive the acquired data from the sensing device, process the received data to determine the coordinates of the robotic arm and the end effector, instruct the robotic arm to move so as to locate the operative portion of the end effector to a desired location relative to the patient and then the end effector to provide the treatment according to the treatment plan.

In one embodiment, the controller is a computer system, a control console, or a microcontroller unit (MCU).

In one embodiment, the treatment plan defines a series of prescribed treatments in which each treatment comprises treatment segments over the skin of the patient and treatment parameters for that treatment segments.

In one embodiment, the treatment parameters for the microneedle treatment tool of the end effector comprise a heat or temperature setting, a choice of red or blue photodynamic therapy, a depth for the microneedles to be inserted, a duration of the treatment, a number of passes for a particular treatment segment, and/or a frequency of the RF energy to be applied.

In another aspect, the invention relates to a method for treating a patient using the above disclosed robotic system. The method comprises:

scanning a portion of the patient to produce data defining a mesh of surfaces between scanned points that corresponds to the skin surface of the scanned portion of the patient, and deriving rendered views of the portion of the patient from the mesh of surfaces, wherein the portion of the patient includes at least areas for the treatment, the rendered views of the portion of the patient are viewable from any desired angle of view, and the rendered views of the portion of the patient serve as a mesh model coordinate system;

identifying the areas for the treatment in the rendered views of the portion of the patient, and dividing the identified areas for the treatment into treatment segments;

identifying the treatment segments in the rendered views of the portion of the patient, and defining treatment parameters for each treatment segment, wherein the treatment segments and treatment parameters collectively constitute the treatment plan to be executed by the robotic system to provide the treatment to the patient;

calibrating the robotic arm with the end effector to set its location in a three-dimensional (3D) spatial coordinate system that serves as a would coordinate system in which the controller determines instructions for movements of the robotic arm and the end effector for the treatment procedure;

calibrating the first and second sensing units to correspond their video and/or distance output from LiDAR sensing to the world coordinate system by a first transformation matrix that converts points in a LiDAR coordinate system into points in the world coordinate system;

locating the portion of the patient in the world coordinate system from the output of the first and second sensing units, and calibrating the relationship between the image of the scanned data of the portion of the patient to the location of the portion of the patient as located in the LiDAR video and distance scan data as a second transformation matrix that converts points in the mesh model coordinate system to points of the actual location of the portion of the patient in the LiDAR coordinate system; and

performing treatment procedure according to the treatment plan by directing the robotic arm to move in a trajectory path in which the end effector is applied to a predetermined series of the treatment segments with the treatment parameters of the treatment plan, and instructing the operative portion of the end effector when in place in each treatment segment to effectuate the treatment for that segment.

The treatment procedure continues until completed for all the treatment segments in the treatment plan.

In one embodiment, the treatment parameters for the microneedle treatment tool of the end effector comprise a heat or temperature setting, a choice of red or blue photodynamic therapy, a depth for the microneedles to be inserted, a duration of the treatment, a number of passes for a particular treatment segment, and/or a frequency of the RF energy to be applied.

In one embodiment, the treatment proceeds only when the load sensor determines that the force along the tool z-axis is above a stamp threshold force, but below maximum values.

In one embodiment, when the treatment proceeds, the microneedles are extended to penetrate the skin of the patient, and to provide the treatment according to the treatment parameters for that treatment segment.

In one embodiment, as the treatment procedure proceeds, in whichever order the treatment segments are addressed, the controller determines a next location for the end effector to be moved to and its orientation, which is the center point of the treatment segment, and wherein the coordinates of that point in the mesh model coordinate system are multiplied by the second transformation matrix to yield the current location in the LiDAR coordinate system, which in turn are multiplied by the first transformation matrix to yield the world coordinates and the robotic arm is directed to move to a place to apply the end effector to that point.

In one embodiment, the method further comprises monitoring a movement of the patient during the treatment procedure, wherein the LiDAR continuously monitors and updates the position of the head of the patient and the second transformation matrix defining the relationship between the mesh model coordinates and the real location of the head coordinates in the LiDAR coordinate system, compensating for any movement of the patient.

In one embodiment, the method further comprises checking processes during the treatment procedure to address situations where the patient moves rapidly, or where anomalous forces on the end effector develop, and, responsive to detection of movement or forces above predetermined thresholds, the data defining the location of the patient's treatment areas in the world coordinate system is updated, or in appropriate situations the robot arm withdraws the end effector from the patient.

In one embodiment, the method further comprises withdrawing the end effector to a safe distance from the patient, when:

a prescribed treatment at a treatment segment is finished;

an average velocity of landmark points on the portion of the patient exceeds a threshold value of a maximally permitted average velocity;

the load cell detects a force in the z-axis of the end effector that exceeds a predetermined maximum value;

the load cell detects a force above another threshold in the x- and or y-axis; or

the operator presses an emergency stop button that is given to the patient.

In yet another aspect, the invention relates to a computerized device for controlling a robotic system in a medical procedure performed on a patient, comprising at least one processor; and a memory device couple to the at least processor, the memory device containing a set of instructions which, when executed by the at least one processor, cause the robotic system to perform a method for treating the patient. The method comprises:

scanning a portion of the patient to produce data defining a mesh of surfaces between scanned points that corresponds to the skin surface of the scanned portion of the patient, and deriving rendered views of the portion of the patient from the mesh of surfaces, wherein the portion of the patient includes at least areas for the treatment, the rendered views of the portion of the patient are viewable from any desired angle of view, and the rendered views of the portion of the patient serve as a mesh model coordinate system;

identifying the areas for the treatment in the rendered views of the portion of the patient, and dividing the identified areas for the treatment into treatment segments;

identifying the treatment segments in the rendered views of the portion of the patient, and defining treatment parameters for each treatment segment, wherein the treatment segments and treatment parameters collectively constitute the treatment plan to be executed by the robotic system to provide the treatment to the patient;

calibrating the robotic arm with the end effector to set its location in a three-dimensional (3D) spatial coordinate system that serves as a would coordinate system in which the controller determines instructions for movements of the robotic arm and the end effector for the treatment procedure;

calibrating the first and second sensing units to correspond their video and/or distance output from LiDAR sensing to the world coordinate system by a first transformation matrix that converts points in a LiDAR coordinate system into points in the world coordinate system;

locating the portion of the patient in the world coordinate system from the output of the first and second sensing units, and calibrating the relationship between the image of the scanned data of the portion of the patient to the location of the portion of the patient as located in the LiDAR video and distance scan data as a second transformation matrix that converts points in the mesh model coordinate system to points of the actual location of the portion of the patient in the LiDAR coordinate system; and

performing treatment procedure according to the treatment plan by directing the robotic arm to move in a trajectory path in which the end effector is applied to a predetermined series of the treatment segments with the treatment parameters of the treatment plan, and instructing the operative portion of the end effector when in place in each treatment segment to effectuate the treatment for that segment.

The treatment procedure continues until completed for all the treatment segments in the treatment plan.

In a further aspect, the invention relates to a non-transitory tangible computer-readable medium storing instructions which, when executed by at least one processor, cause a robotic system to perform a method for treating a patient. The method comprises:

scanning a portion of the patient to produce data defining a mesh of surfaces between scanned points that corresponds to the skin surface of the scanned portion of the patient, and deriving rendered views of the portion of the patient from the mesh of surfaces, wherein the portion of the patient includes at least areas for the treatment, the rendered views of the portion of the patient are viewable from any desired angle of view, and the rendered views of the portion of the patient serve as a mesh model coordinate system;

identifying the areas for the treatment in the rendered views of the portion of the patient, and dividing the identified areas for the treatment into treatment segments;

identifying the treatment segments in the rendered views of the portion of the patient, and defining treatment parameters for each treatment segment, wherein the treatment segments and treatment parameters collectively constitute the treatment plan to be executed by the robotic system to provide the treatment to the patient;

calibrating the robotic arm with the end effector to set its location in a three-dimensional (3D) spatial coordinate system that serves as a would coordinate system in which the controller determines instructions for movements of the robotic arm and the end effector for the treatment procedure;

calibrating the first and second sensing units to correspond their video and/or distance output from LiDAR sensing to the world coordinate system by a first transformation matrix that converts points in a LiDAR coordinate system into points in the world coordinate system;

locating the portion of the patient in the world coordinate system from the output of the first and second sensing units, and calibrating the relationship between the image of the scanned data of the portion of the patient to the location of the portion of the patient as located in the LiDAR video and distance scan data as a second transformation matrix that converts points in the mesh model coordinate system to points of the actual location of the portion of the patient in the LiDAR coordinate system; and

performing treatment procedure according to the treatment plan by directing the robotic arm to move in a trajectory path in which the end effector is applied to a predetermined series of the treatment segments with the treatment parameters of the treatment plan, and instructing the operative portion of the end effector when in place in each treatment segment to effectuate the treatment for that segment.

The treatment procedure continues until completed for all the treatment segments in the treatment plan.

These and other aspects of the present invention will become apparent from the following description of the preferred embodiments, taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. The same reference numbers may be used throughout the drawings to refer to the same or like elements in the embodiments.

FIG. 1 shows schematically a robotic system according to embodiments of the invention.

FIG. 2 is a perspective view of an operative part of an end effector, according to embodiments of the invention, where the end effector is a microneedle RF device.

FIG. 3 is a front end view of the microneedle end effector shown in FIG. 2.

FIG. 4 is a diagram showing the components of the medical robot system according to embodiments of the invention.

FIG. 5 is a diagram of one type of scanner for taking the three dimensional scan of a patient to be treated.

FIG. 6 a perspective view of another scanning device that may be used to derive a three dimensional scan of a patient's face according to embodiments of the invention.

FIG. 7 is a rendered perspective view showing the surface defined by the data points derived by scanning the face of a patient.

FIG. 8 is another rendered view of the scanned points of the patient's face from a different viewing angle.

FIG. 9 is front rendered view of the patient's scan as displayed on a computer monitor by a software program tool used for defining a treatment plan for applying treatment areas.

FIG. 10 is a view as seen in FIG. 9, showing square treatment areas defined by a user with the software tool, defining the treatment plan for the patient.

FIG. 11 is another view of a rendering of the high-definition (hi-def) scanned mesh model of the patient's head with treatment segments defined thereon.

FIG. 12 is another view of a rendering as in FIG. 11, but rendered from a different viewpoint.

FIG. 13 is a screenshot of the interactive software tool for preparing the facial mapping and the parameters of the treatment plan for each of the square treatment segments of the patient.

FIG. 14 is a view of a patient during a treatment procedure.

FIG. 15 is a detail diagram of the microneedle end effector approaching a treatment segment.

FIG. 16 is a detail diagram as in FIG. 15, but where the microneedle end effector is aligned with the normal to the centerpoint of the treatment segment.

FIG. 17 is a detail diagram as in FIG. 16, but where the microneedle end effector has engaged the skin of the patient.

FIG. 18 is a view of an alternate embodiment of LiDAR and cameras viewed looking downward from the top of the bed and over the head of the patient.

FIG. 19 is an exemplary view of a camera image of a person that was processed by Google FaceMesh so as to locate the face of the person and landmark points in the face.

FIG. 20 is an exemplary view of a rendering of the hi-def mesh model of the scan of the same person as in FIG. 9, also processed by Google FaceMesh so as to locate the face and landmark points in the face.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a”, “an”, and “the” includes plural reference unless the context clearly dictates otherwise. Also, it will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this invention, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present invention, and will not be interpreted in an idealized or overly formal sense unless expressly so defined herein.

The following description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. For purposes of clarity, the same reference numbers will be used in the drawings to identify similar elements. As used herein, the phrase at least one of A, B, and C should be construed to mean a logical (A or B or C), using a non-exclusive logical OR. It should be understood that one or more steps within a method may be executed in different order (or concurrently) without altering the principles of the present disclosure.

LiDAR, also Lidar or LIDAR, is an acronym of “light detection and ranging” or “laser imaging, detection, and ranging”. It is sometimes called 3-D laser scanning, a special combination of 3-D scanning and laser scanning. LiDAR is a method for determining ranges (variable distance) by targeting an object or a surface with a laser and measuring the time for the reflected light to return to the receiver.

As used herein, the term module may refer to, be part of, or include an Application Specific Integrated Circuit (ASIC); an electronic circuit; a combinational logic circuit; a field programmable gate array (FPGA); a processor (shared, dedicated, or group) that executes code; other suitable hardware components that provide the described functionality; or a combination of some or all of the above, such as in a system-on-chip. The term module may include memory (shared, dedicated, or group) that stores code executed by the processor.

The term code may include software, firmware, and/or microcode, and may refer to programs, routines, functions, classes, and/or objects. The term shared, as used above, means that some or all code from multiple modules may be executed using a single (shared) processor. In addition, some or all code from multiple modules may be stored by a single (shared) memory. The term group, as used above, means that some or all code from a single module may be executed using a group of processors. In addition, some or all code from a single module may be stored using a group of memories.

The apparatuses and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system. By way of example, an element, or any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more example embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. It should be understood that one or more steps within a method may be executed in different order (or concurrently) without altering the principles of the invention.

This invention to provide computer controlled systems and methods for performing medical operations where a computer controls the movement of a robotic arm with an end effector that is a medical tool interacting with the patient. The computer controlled systems are capable of performing operations on a patient without substantial amounts of human control and where the patient moves.

In one aspect, the invention relates to a robotic system for treating a patient. The robotic system comprises a robotic arm with an end effector for treating the patient, wherein the robotic arm is configured to be movable in a space surrounding the patient, and the end effector is configured to be movable individually and co-movable with the robotic arm in said space; a sensing device for acquiring data associating with coordinates and images of the end effector and the patient; and a controller in communications with the robotic arm and the sensing device for controlling movements of the robotic arm with the end effector and treatments of the patient with the end effector based on the acquired data and a treatment plan.

In one embodiment, the end effector is supported on a load sensor on the end of the robotic arm so that the end effector is movable by the robotic arm to essentially any location in said space.

In one embodiment, the load sensor is a three-axis load sensor for sensing the forces being applied to the end effector in three orthogonal axes, x, y and z, with the z-axis being the lengthwise axis of the end effector and the x- and y-axes being lateral dimensions perpendicular to that vertical dimension and each other.

In one embodiment, the end effector comprises an operative portion that acts on the patient directly, a tool control circuit for controlling actions of the end effector, and a housing containing the electronic circuitry, wherein a proximal end of a housing is supported on the load sensor, and a distal end of the housing supports the operative portion.

In one embodiment, the end effector is a surgical instrument or a medical instrument.

In one embodiment, the end effector is a scalpel, scissors, an electrocauterizer, a gas plasma treatment tool, and/or a microneedle treatment tool.

In one embodiment, the microneedle treatment tool comprises an array of microneedles configured such that each microneedle is selectively activatable to extend into or retract from the skin of the patient independently.

In one embodiment, the microneedle treatment tool is further configured to apply radio frequency (RF) waveforms, heat, light, and/or drug through the array of microneedles to the skin of the patient for therapy.

In one embodiment, the array of microneedles are supported on a structure in the end effector that selectively extends them out through a planar front face of the end effector and into the skin of the patient, or retracts them back behind the planar front face.

In one embodiment, a force applied to each microneedles to enter the skin of the patient is selected at varied levels.

In one embodiment, the sensing device comprises a first sensing unit and a second sensing unit, wherein the first sensing unit is disposed in said space at a stationary location vertically above the patient and directed at the patient, and the second sensing unit is attached on the end effector, such that during the treatment, the second sensing unit moves with the end effector, while the first sensing remains stationary on its support over the patient.

In one embodiment, each of the first and second sensor units comprises a LiDAR sensor and at least one camera, wherein the LiDAR sensor is configured to determine distances of the LiDAR sensor to surfaces of objects in its field of view, and the at least one camera is configured to acquire stereoscopic images of the objects in its field of view.

In one embodiment, the acquired data by each sensing unit comprises an array of range data, and video data for a field of pixels, wherein the range data for each pixel is an optically derived LiDAR distance value of the distance from the LiDAR sensor to the nearest object met by a ray extending through that pixel from the LiDAR sensor.

In one embodiment, the sensing device comprises a LiDAR sensor located in a center of the bed above the patient; and a first stereoscopic camera and a second stereoscopic camera symmetrically located a distance to the left and right of the LiDAR sensor, respectively, wherein the LiDAR sensor and the first and second stereoscopic cameras are attached on a stationary support.

In one embodiment, the controller is in wired or wireless communications with the robotic arm and the sensing device. In one embodiment, the data transmissions between the controller and the robotic arm and the sensing device can be performed through any wireless communication networks and protocols, such as, but are not limited to, Bluetooth, Wi-Fi, Near Field Communication (NFC) protocols, or the likes.

In one embodiment, the controller is configured to receive the acquired data from the sensing device, process the received data to determine the coordinates of the robotic arm and the end effector, instruct the robotic arm to move so as to locate the operative portion of the end effector to a desired location relative to the patient and then the end effector to provide the treatment according to the treatment plan.

In one embodiment, the controller is a computer system, a control console, or a microcontroller unit (MCU).

In one embodiment, the treatment plan defines a series of prescribed treatments in which each treatment comprises treatment segments over the skin of the patient and treatment parameters for that treatment segments.

In one embodiment, the treatment parameters for the microneedle treatment tool of the end effector comprise a heat or temperature setting, a choice of red or blue photodynamic therapy, a depth for the microneedles to be inserted, a duration of the treatment, a number of passes for a particular treatment segment, and/or a frequency of the RF energy to be applied.

In another aspect, the invention relates to a method for treating a patient using the above disclosed robotic system. The method comprises:

scanning a portion of the patient to produce data defining a mesh of surfaces between scanned points that corresponds to the skin surface of the scanned portion of the patient, and deriving rendered views of the portion of the patient from the mesh of surfaces, wherein the portion of the patient includes at least areas for the treatment, the rendered views of the portion of the patient are viewable from any desired angle of view, and the rendered views of the portion of the patient serve as a mesh model coordinate system;

identifying the areas for the treatment in the rendered views of the portion of the patient, and dividing the identified areas for the treatment into treatment segments;

identifying the treatment segments in the rendered views of the portion of the patient, and defining treatment parameters for each treatment segment, wherein the treatment segments and treatment parameters collectively constitute the treatment plan to be executed by the robotic system to provide the treatment to the patient;

calibrating the robotic arm with the end effector to set its location in a three-dimensional (3D) spatial coordinate system that serves as a would coordinate system in which the controller determines instructions for movements of the robotic arm and the end effector for the treatment procedure;

calibrating the first and second sensing units to correspond their video and/or distance output from LiDAR sensing to the world coordinate system by a first transformation matrix that converts points in a LiDAR coordinate system into points in the world coordinate system;

locating the portion of the patient in the world coordinate system from the output of the first and second sensing units, and calibrating the relationship between the image of the scanned data of the portion of the patient to the location of the portion of the patient as located in the LiDAR video and distance scan data as a second transformation matrix that converts points in the mesh model coordinate system to points of the actual location of the portion of the patient in the LiDAR coordinate system; and

performing treatment procedure according to the treatment plan by directing the robotic arm to move in a trajectory path in which the end effector is applied to a predetermined series of the treatment segments with the treatment parameters of the treatment plan, and instructing the operative portion of the end effector when in place in each treatment segment to effectuate the treatment for that segment,

The treatment procedure continues until completed for all the treatment segments in the treatment plan.

In one embodiment, the treatment parameters for the microneedle treatment tool of the end effector comprise a heat or temperature setting, a choice of red or blue photodynamic therapy, a depth for the microneedles to be inserted, a duration of the treatment, a number of passes for a particular treatment segment, and/or a frequency of the RF energy to be applied.

In one embodiment, the treatment proceeds only when the load sensor determines that the force along the tool z-axis is above a stamp threshold force, but below maximum values.

In one embodiment, when the treatment proceeds, the microneedles are extended to penetrate the skin of the patient, and to provide the treatment according to the treatment parameters for that treatment segment.

In one embodiment, as the treatment procedure proceeds, in whichever order the treatment segments are addressed, the controller determines a next location for the end effector to be moved to and its orientation, which is the center point of the treatment segment, and wherein the coordinates of that point in the mesh model coordinate system are multiplied by the second transformation matrix to yield the current location in the LiDAR coordinate system, which in turn are multiplied by the first transformation matrix to yield the world coordinates and the robotic arm is directed to move to a place to apply the end effector to that point.

In one embodiment, the method further comprises monitoring a movement of the patient during the treatment procedure, wherein the LiDAR continuously monitors and updates the position of the head of the patient and the second transformation matrix defining the relationship between the mesh model coordinates and the real location of the head coordinates in the LiDAR coordinate system, compensating for any movement of the patient.

In one embodiment, the method further comprises checking processes during the treatment procedure to address situations where the patient moves rapidly, or where anomalous forces on the end effector develop, and, responsive to detection of movement or forces above predetermined thresholds, the data defining the location of the patient's treatment areas in the world coordinate system is updated, or in appropriate situations the robot arm withdraws the end effector from the patient.

In one embodiment, the method further comprises withdrawing the end effector to a safe distance from the patient, when:

a prescribed treatment at a treatment segment is finished;

an average velocity of landmark points on the portion of the patient exceeds a threshold value of a maximally permitted average velocity;

the load cell detects a force in the z-axis of the end effector that exceeds a predetermined maximum value;

the load cell detects a force above another threshold in the x- and or y-axis; or

the operator presses an emergency stop button that is given to the patient.

In yet another aspect, the invention relates to a computerized device for controlling a robotic system in a medical procedure performed on a patient, comprising at least one processor; and a memory device couple to the at least processor, the memory device containing a set of instructions which, when executed by the at least one processor, cause the robotic system to perform a method for treating the patient. The method comprises:

scanning a portion of the patient to produce data defining a mesh of surfaces between scanned points that corresponds to the skin surface of the scanned portion of the patient, and deriving rendered views of the portion of the patient from the mesh of surfaces, wherein the portion of the patient includes at least areas for the treatment, the rendered views of the portion of the patient are viewable from any desired angle of view, and the rendered views of the portion of the patient serve as a mesh model coordinate system;

identifying the areas for the treatment in the rendered views of the portion of the patient, and dividing the identified areas for the treatment into treatment segments;

identifying the treatment segments in the rendered views of the portion of the patient, and defining treatment parameters for each treatment segment, wherein the treatment segments and treatment parameters collectively constitute the treatment plan to be executed by the robotic system to provide the treatment to the patient;

calibrating the robotic arm with the end effector to set its location in a three-dimensional (3D) spatial coordinate system that serves as a would coordinate system in which the controller determines instructions for movements of the robotic arm and the end effector for the treatment procedure;

calibrating the first and second sensing units to correspond their video and/or distance output from LiDAR sensing to the world coordinate system by a first transformation matrix that converts points in a LiDAR coordinate system into points in the world coordinate system;

locating the portion of the patient in the world coordinate system from the output of the first and second sensing units, and calibrating the relationship between the image of the scanned data of the portion of the patient to the location of the portion of the patient as located in the LiDAR video and distance scan data as a second transformation matrix that converts points in the mesh model coordinate system to points of the actual location of the portion of the patient in the LiDAR coordinate system; and performing treatment procedure according to the treatment plan by directing the robotic arm to move in a trajectory path in which the end effector is applied to a predetermined series of the treatment segments with the treatment parameters of the treatment plan, and instructing the operative portion of the end effector when in place in each treatment segment to effectuate the treatment for that segment.

The treatment procedure continues until completed for all the treatment segments in the treatment plan.

In a further aspect, the invention relates to a non-transitory tangible computer-readable medium storing instructions which, when executed by at least one processor, cause a robotic system to perform a method for treating a patient. The method comprises:

scanning a portion of the patient to produce data defining a mesh of surfaces between scanned points that corresponds to the skin surface of the scanned portion of the patient, and deriving rendered views of the portion of the patient from the mesh of surfaces, wherein the portion of the patient includes at least areas for the treatment, the rendered views of the portion of the patient are viewable from any desired angle of view, and the rendered views of the portion of the patient serve as a mesh model coordinate system;

identifying the areas for the treatment in the rendered views of the portion of the patient, and dividing the identified areas for the treatment into treatment segments;

identifying the treatment segments in the rendered views of the portion of the patient, and defining treatment parameters for each treatment segment, wherein the treatment segments and treatment parameters collectively constitute the treatment plan to be executed by the robotic system to provide the treatment to the patient;

calibrating the robotic arm with the end effector to set its location in a three-dimensional (3D) spatial coordinate system that serves as a would coordinate system in which the controller determines instructions for movements of the robotic arm and the end effector for the treatment procedure;

calibrating the first and second sensing units to correspond their video and/or distance output from LiDAR sensing to the world coordinate system by a first transformation matrix that converts points in a LiDAR coordinate system into points in the world coordinate system;

locating the portion of the patient in the world coordinate system from the output of the first and second sensing units, and calibrating the relationship between the image of the scanned data of the portion of the patient to the location of the portion of the patient as located in the LiDAR video and distance scan data as a second transformation matrix that converts points in the mesh model coordinate system to points of the actual location of the portion of the patient in the LiDAR coordinate system; and performing treatment procedure according to the treatment plan by directing the robotic arm to move in a trajectory path in which the end effector is applied to a predetermined series of the treatment segments with the treatment parameters of the treatment plan, and instructing the operative portion of the end effector when in place in each treatment segment to effectuate the treatment for that segment.

The treatment procedure continues until completed for all the treatment segments in the treatment plan.

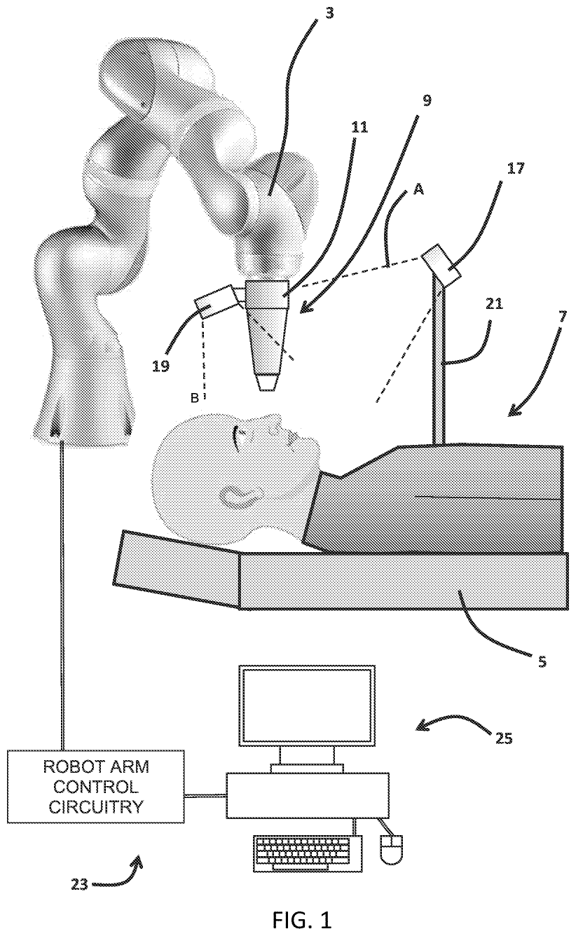

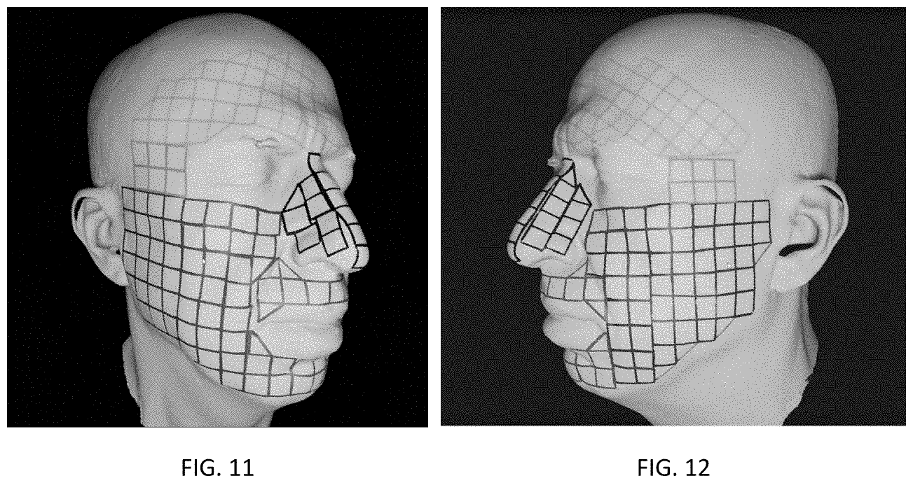

Certain exemplary embodiments of the invention are described as follows, in conjunction with the accompanying drawings of FIGS. 1-20. For purposes of clarity, the same reference numbers will be used in the drawings to identify similar elements.

As best shown in FIG. 1, a robotic arm 3 is supported adjacent an operating bed 5 on which a patient indicated generally at 7 is lying. The bed allows for the patient to lie supine if appropriate for the procedure, but the robotic arm 3 is generally agnostic as to the patient orientation, and the patient may alternatively be supported in a chair or some other supporting device well known in the medical art.

The robotic arm 3 is provided at its movable end with an end effector or medical instrument indicated at 9. The end effector 9 is supported on a load-sensor apparatus (e.g., load cell) 11 on the end of the robotic arm 3 so that the end effector 9 may be moved by the robotic arm 3 to essentially any location in the space surrounding the robotic arm 3.

A proximal end of a housing 13 is supported on the load sensor apparatus 11, and the housing 13 contains electronic circuitry controlling actions of the end effector 9. The distal end of the housing 13 supports the operative portion 15 of the end effector 9 that acts on the patient directly. In the preferred embodiment, the operative portion 15 of the end effector 9 is a microneedle apparatus configured to engage the skin of a patient for facial and medical treatments, as will be discussed below.

A support 21 adjacent or attached to the bed 5 holds a LiDAR and stereoscopic apparatus (i.e., LiDAR camera system/device) 17 above the patient at a stationary location vertically above the patient at approximately his or her lateral center line at a height of about 0.5 to 1.5 meters above the bed. The LiDAR and camera system 17 is directed at the patient, and the LiDAR transmits 860 nm infrared radiation to determine distance of the LiDAR sensor to the surface of the patient or of any other objects in the field of view, e.g., the end effector 9 or the robotic arm 3 and other structure in the operating theatre that is in front of the LiDAR camera system 17. The camera part of the LiDAR camera system 17 takes color high-definition (hi-def) stereoscopic visual imagery and outputs electronic data corresponding to sequential color frames of the imagery. The LiDAR camera system 17 also continually outputs electronic data corresponding to an array of LiDAR-based distance data over its field of view, which is indicated by the phantom lines A.

A second similar LiDAR camera system 19 is supported on the end effector assembly 9, positioned upward relative to the patient from the actual operative portion 15 of the end effector 9. The second LiDAR camera system 19 moves with the end effector 9 during the treatment, while the LiDAR camera system 17 remains stationary on its support 21 over the patient 7. The movable LiDAR camera system 19 has a field of view, indicated by phantom lines B, that moves with the end effector 9, and within the field of view returns color hi-def video of the patient from two laterally spaced cameras, as well as an array of data of optically derived LiDAR distance values of the distance from the LiDAR sensor 19 to points on any objects in front of the LiDAR sensor 19 as it is moved with the end effector 9.

The robotic arm 3 is electrically powered and its movement is controlled electrically by robot arm circuitry 23 connected to it. The robot arm control circuitry 23 is electrically connected with a computer system generally indicated at 25, which is preferably a PC computer system, as well known in the art, having a computer with one or more processors, internal memory, mass storage devices such as discs or other types of memory, a hi-def color monitor, a keyboard, a mouse, and any other peripherals that may be desirable in this context. The computer system 25 controls all operations of the robotic arm 3 by instructions through the robotic arm control circuitry 23. The computer system 25 is also coupled with the LiDAR and stereoscopic camera systems 17 and 19 so as to receive data corresponding to the visual imagery of the cameras of those devices, and the distance data from the LiDAR sensors in those units 17 and 19.

In some embodiments, the data transmissions between the computer system 25 and the robotic arm 3 and the LiDAR and stereoscopic camera systems 17 and 19 are performed through wired communications, by, for example, wire connecting the computer system 25 to the robotic arm 3 and/or the LiDAR and stereoscopic camera systems 17 and 19.

In another embodiments, the data transmissions between the computer system 25 and the robotic arm 3 and the LiDAR and stereoscopic camera systems 17 and 19 are performed through any wireless communication networks and protocols, such as, but are not limited to, Bluetooth, Wi-Fi, Near Field Communication (NFC) protocols, or the likes. In such embodiments, each of the computer system 25 and the robotic arm 3 and the LiDAR and stereoscopic camera systems 17 and 19 may include one or more wireless transceiver modules for receiving and transmitting data therebetween wirelessly.

The robotic arm 3 in one embodiment is a robot arm manufactured by the company KUKA AG and designated the LBR iiwa 7 R800 medical model. The robotic arm is sold together with a cabinet and a “SmartPad” that allows for manual control of the robotic arm 3 by a human user, as well as the control circuitry 23 that allows the computer system 25 connected with the control circuitry 23 to send electrical signals comprising commands that are interpreted by the robot control circuitry 23 to cause the robotic arm 3 to move to specified locations with a high degree of accuracy, i.e., less than 1 mm, with the repeatability of the robotic arm 3 of the preferred embodiment being ±0.1 mm to ±0.15 mm. It should be noted that other robotic arms can also be utilized to practice the invention.

Control software provided with the robotic arm 3 by the manufacturer controls movement of the robotic arm 3, and the software is configured to provide for an interface with the computer system 25 that uses the software to prepare and transmit electrical signal commands to the robotic arm 3 so as to direct precise controlled movement of the robotic arm 3. In addition, the robotic arm 3 provides feedback top the computer system 25 defining the exact current location of the end effector 9 and its orientation in a world coordinate system of the robotic arm 3, as will be discussed herein.

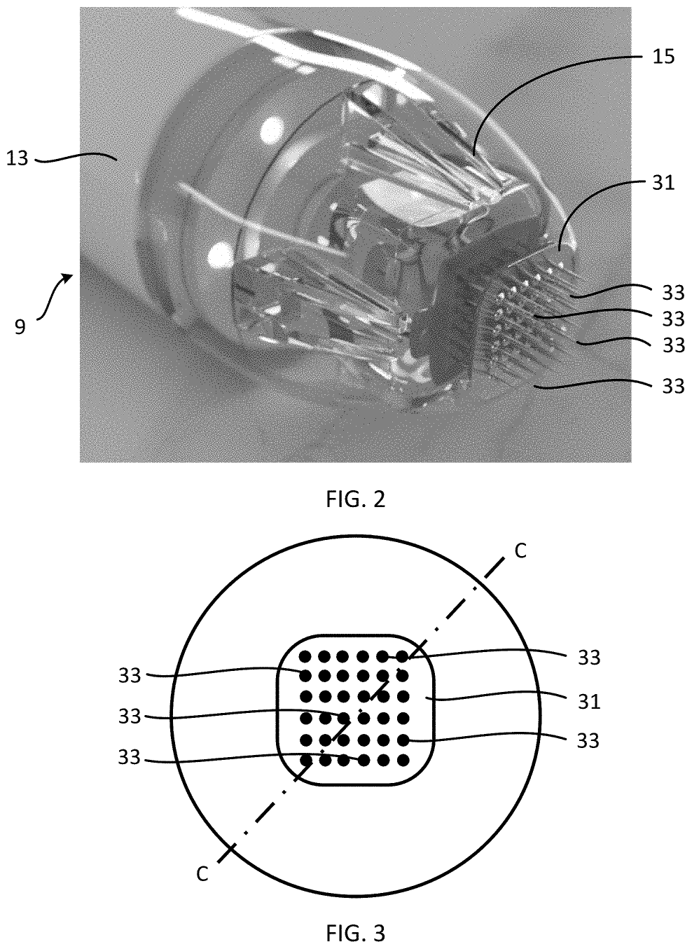

The medical tool attached to the robotic arm 3 as an end effector 9 may be any tool used in medical applications, but, in the preferred embodiment shown in FIGS. 2-3, the end effector 9 supported by housing 13 is a microneedle tool. The operative part of the microneedle tool/device 15 is similar to a handheld microneedle RF treatment tool sold by Aesthetics Biomedical Inc. under the name of Vivace.

The operative end portions of both the manual tool and the robotic arm tool are similar in that they both have the same square-matrix array of 36 or more parallel gold needles 33 organized in rows and columns within a square perimeter of approximately 1 cm. The needles 33 are supported to selectively extend out through holes in a planar, roughly square, end face 31 of the end effector 9. The needles 33 are supported on a structure in the end effector 9 that selectively extends them out through the planar end-surface of the end effector, as shown in FIG. 2, and into the skin of the patient, or retracts them together back behind the planar end face 31.

The microneedle tool/device is used by placing it perpendicularly against the surface of the patient's skin and then activating the needles so they move forward and enter into the skin of the patient. An alternating RF frequency electrical current then is transmitted through the needles, which stimulates the patient's skin tissue and provides a therapeutic effect on the skin of the patient so treated.

In the handheld tool used by manual treatment personnel, all of the needles are actuated together. The microneedle tool 15 of the embodiment shown here can activate all of the needles to extend together simultaneously, as in the handheld tool, or a subset of the needles may be selectively activated to extend forward, while the remainder of the needles remains retracted. Preferably, the smaller subset of the matrix of needles is a triangular set of the needles, such as the triangular set of needles on one side of the diagonal line C-C in FIG. 3. Also, it is possible to configure the microneedle tool 15 so that each needle 33 is selectively extended and retracted independently with computer control.

Furthermore, the force applied to the needles 33 to enter the patient's skin may be selected at varied levels by the electronic control of the tool. In addition, the tool end 15 may also provide for the application of heat and/or light to the area in front of the planar front face 31 of the end effector. All of those parameters of the function of the operative portion 15 of the end effector 9 are controlled electronically by the computer system 25.

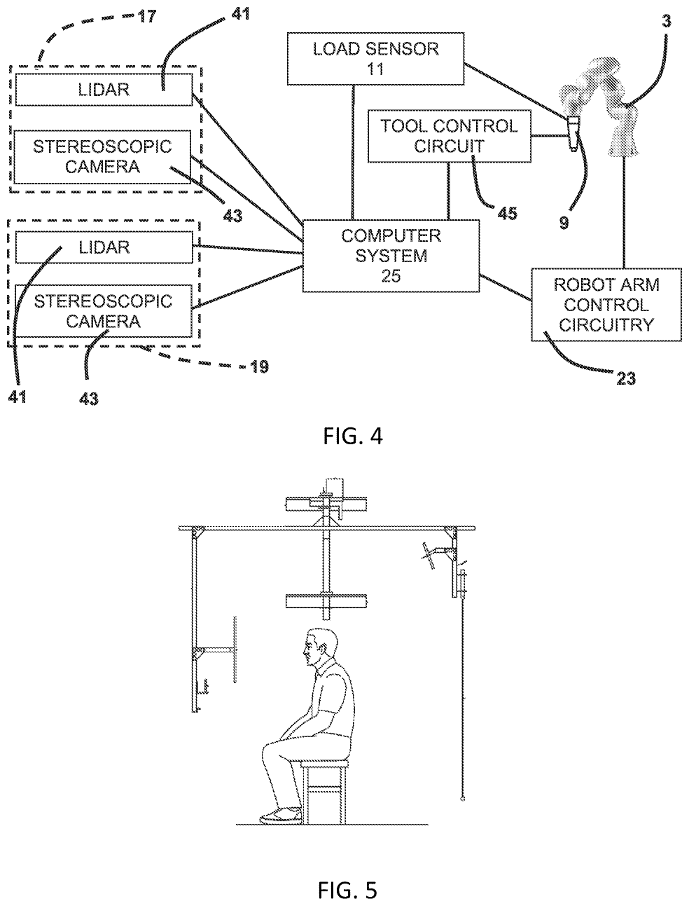

FIG. 4 shows the relevant components and associated peripherals of the robotic control system.

The computer system 25 receives inputs from both the LiDAR component 41 and the camera component 43 of each of the LiDAR camera systems/units 17 and 19. The sensor unit 17 is mounted stationary on a support structure such as a typical surgical mount well-known in the art adjacent the operating bed, and the other sensor unit 19 is mounted via a connection to the support structure (load sensor) 11 on a mounting flange of the end effector 9. Both sensor units 17 and 19 are preferably the same type of device, e.g., the combination camera and LiDAR system sold by Intel under the designation Intel RealSense camera L515. This device outputs a LiDAR data signal corresponding to a data array for a field of pixels, wherein each pixel is data defining the distance to the nearest object met by a ray extending through that pixel from the LiDAR sensor. The data field of pixels may range in resolution from low-res, i.e., QVGA (320×240 data points) to preferably high-res, i.e., XGA (1024×786 data points) for the sensor field of view of 70 degrees by 55 degrees. The operating range varies for reflectivity, but is at least 0.25 meter to 2.6 meters and preferably at least to 3.9 meters. The cameras present in the L515 LiDAR sensor also produce RGB color video data for a field of pixels in YUY2 format in resolutions varying from 960×540 to 1920×1080. It should be noted that other LiDAR camera systems can also be utilized to practice the invention.

The range and color video output data from the LiDAR sensors 17 and 19 is transmitted to the computer system 25 for control and monitoring of the robotic arm treatment procedure, as will be set out below.

In addition, the computer system 25 receives an electrical data signal from the load cell 11, which provides digital data indicative of the forces being applied to the end effector 9 in three separate orthogonal axes, x, y and z, with the z-axis being the lengthwise axis of the end effector 9 and the x- and y-axes being lateral dimensions perpendicular to that vertical dimension and each other. The data signal received is a force value in each of those directions, which is continuously transmitted to the computer system 25.

The load cell/sensor 11 in the preferred environment is a 50 newton load cell manufactured by the Forsentek company (www forsentek com). The load sensor is a three-axis load sensor and output stick that outputs analog signals indicative of the force loading in each of the x, y and z directions relative to the robotic arm 3. The output signals are amplified so as to be readable by a micro-controller and a micro-controller such as the micro-controller sold under the identifier STM32F446 by STM Micro Electronics (www.st.com). This microcontroller converts the amplified signal output from the load sensor into a digital data signal and transmits it to the computer system 25. It should be noted that other load cells/sensors can also be utilized to practice the invention.

The computer system 25 uses the information received from the sensors as well as stored data and treatment programs, as will be described below, to formulate and transmit control signals to the robotic arm controlled circuitry 23, which causes the robotic arm to move to locate the operative end of the end effector 9 to the appropriate location relative to the patient's body to provide the treatment set out in a predetermined treatment plan.

In addition to controlling the robotic arm 3, the computer system 25 communicates with a tool control circuit 45 that receives electronic signals for control of the operation of the end effector tool 9 itself. For example, when the end effector tool 9 is a microneedling device, instructions are sent to activate the needles so as to extend into the skin of the patient or to transmit to that patient an RF treatment output, or any other parameters of the tool operation controllable by the computer system.

The use of the robotic arm 3 is not restricted to any part of the human body and may be employed in a variety of surgical, therapeutic or prophylactic treatments. It is believed that robotic medical treatment is particularly desirable and needed for treatment of the face area of a patient, especially with medical treatment of the skin of the face with tools like a microneedle RF tool, and the preferred embodiment shows a robotic medical system according to the invention being applied in that area. This, however, is just an example of one of the ways in which the robotic arm may be utilized, and it will be understood that the system could be applied to the foot or other parts of the human body.

Without intent to limit the scope of the invention, the example of using a robotic medical system for microneedle RF therapy of the facial skin of a patient is given below. Note that titles or subtitles may be used in the example for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Initial Scanning

For this type of treatment for a patient, it is first necessary to obtain a definition of the area of treatment for the patient. This is done by first performing a high resolution scan of the patient. Scanning of this type is well known in the art, especially in the area of film production where actors' faces are scanned for creation of computer-generated imagery (CGI) effects.

For example, in one embodiment one apparatus for such scanning is described in a published patent application U.S. Ser. No. 2016/0353022 A1 of oVio Technologies LLC. An exemplary figure is shown in FIG. 5 herein. A patient is seated in an apparatus and a camera mounted on a room takes a series of photographs or a video as it moves around him. The set of images is processed to produce data defining the exterior surface of the patient's head or upper body.

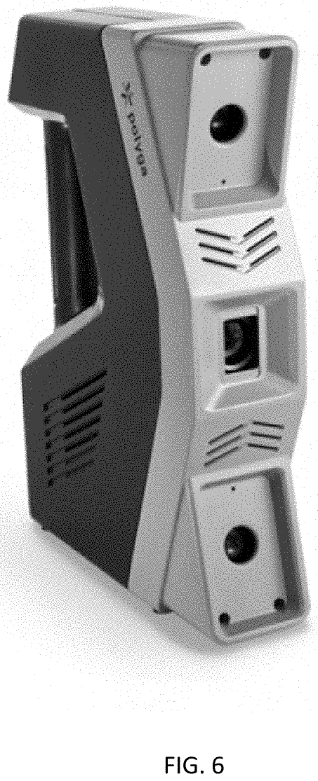

In other embodiments, another device that is preferably used to scan the face of a patient is shown in FIG. 6. The scanner is the H3 3D handheld scanner sold by Polyga. It produces a 3D scan with an accuracy up to 8 microns (i.e., 0.08 mm).

The output of the scan is data stored to be accessible to the computer system 25. The data identifies the points in a spatial coordinate system that constitute the individual points that together make up the scanned portion of the patient. In the computer system 25 or in a computer that is connected with the scanning device, data defining connecting surfaces between the discrete points is determined, resulting data defining a mesh of defined surfaces between the scanned points that corresponds closely to the actual skin surface of the scanned portion of the patient.

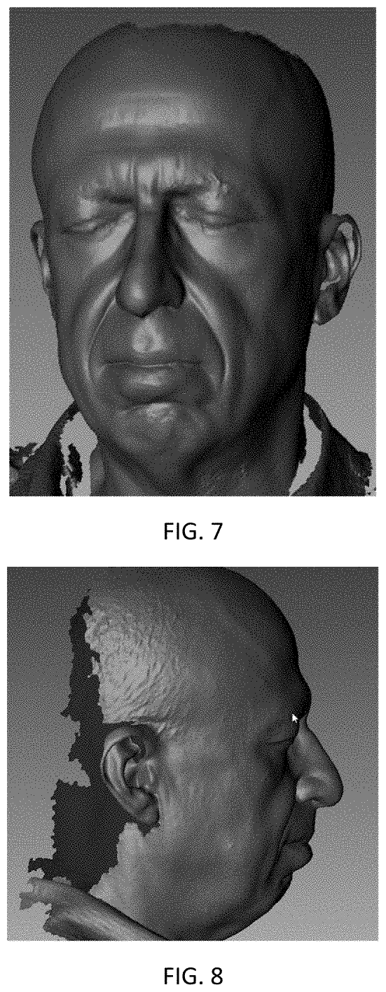

FIGS. 7-8 are rendered views derived from such a mesh of data points. The accuracy of the points is high, i.e., 8 microns, so the details of the surface are clear and defined with great specificity and accuracy. The scan need not be the complete head of the patient, as the absence of the scan of the back of the patient's head visible in FIG. 8 shows. However, the skin of the face of the patient where it is scanned is defined with high accuracy.

### Treatment Segments and Treatment Plan

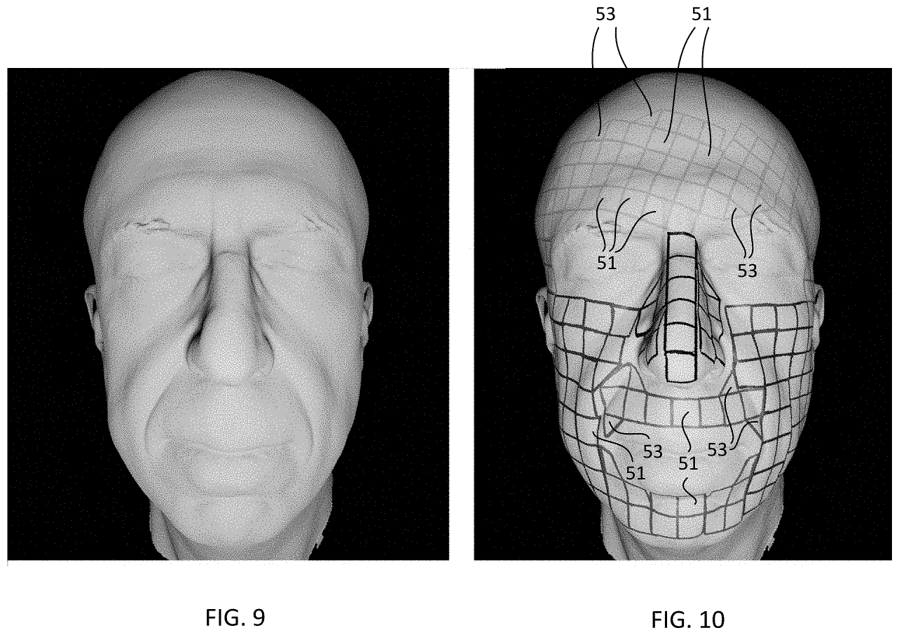

The computer system 25 is equipped with software that provides to a technician setting up the medical robotic treatment the facility to view the scanned face of the patient. The interactive screen of the software tool displays the rendered view of the face from any desired angle of view, i.e., the view of FIG. 7 or 8, or any other view, which can be selected by rotating the image of the head using the mouse pointer. The view may also be a frontal view of the scanned face, as is shown in FIG. 9.

Referring to FIGS. 10-12, the technician, relying on the rendered views of the face from any angle or point of view desired, then proceeds to use the software tool to identify areas of the face where the end effector should be applied. Because the exemplary end effector here is a microneedle device with an operative area that is a square of about 1 cm on each side, the tool allows the user to identify the areas for treatment and divide them up into treatment segments 51, each of which is a 1 cm square. The image can be rotated freely to display all sides of the scan of the face, allowing the user to place segments in any area of the face that is to be treated.

The software tool allows the user to selectively locate the treatment segments 51 wherever desired, generally with the constraint that the area must be a 1 cm square on the face of the patient. However, the user may elect to make the selection only in certain areas, and the user can avoid identifying treatment segments, therefore excluding, areas like the eyes or lips as part of the treatment.

The scanned portion of the patient is a set of points defined in a local three-dimensional coordinate system pertaining only to the scanned set of points and only used internally in the computer system 25. The individual treatment segments 51 are each defined in computer memory as respective data sets that correspond to the coordinates in the local three-dimensional coordinate system of all the four corners of each treatment segment 51, together with the coordinates of the center point in the segment.

Certain areas 53 may also be identified by the technician setting up the segments as triangular where the end effector tool is configured to apply a triangular area of treatment. The identifications of triangular treatment segments are stored in the computer 25 by the coordinates of the three corner points and the coordinates of the center point of the triangular treatment segment 53.

The division of the face up into treatment segments is done manually by a user, but may also be done by a computer calculation, especially an AI program that is trained appropriately.

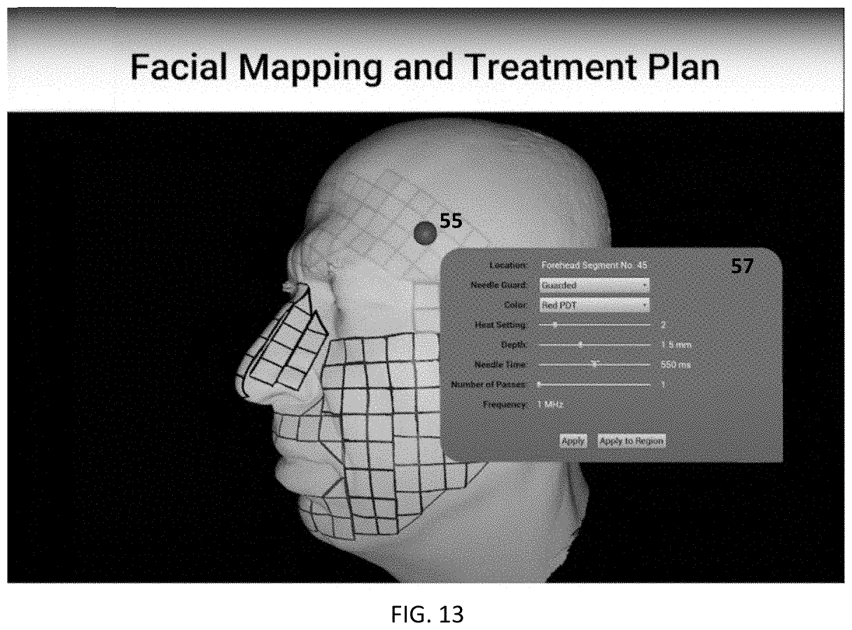

After the treatment segments 51 and 53 are defined on the scanned surface of the patient's face, then the software tool provides an interactive view that allows the technician to define the treatment parameters for each treatment segment as shown in FIG. 13. A pointer in the form of ball 55 is placed on a given treatment segment of the treatment in the rendered view, and a dialog box 57 opens, allowing the user to enter the treatment parameters for that treatment segment 51. The treatment parameters for the microneedle tool end effector may be a heat or temperature setting, a choice of red or blue photodynamic therapy, a depth for the needles to be inserted, the duration of the treatment, the number of passes for the particular segment, and the frequency of the RF energy to be applied. This feature allows flexible, customized variation of the treatment over the face of the patient.

### Treatment Procedure

After the treatment segments are identified and the treatment parameters for each segment are defined, the collective data constitutes a treatment plan that can be executed by the computer system 25 to provide the treatment to the patient.

Generally, the treatment procedure involves first calibrating the robotic arm 3 with the end effector 9 to set its location in a three-dimensional spatial coordinate system that serves as the coordinate system in which the computer system 25 determines the instructions for movement of the robotic arm 3 and the end effector 9 for the treatment procedure. This coordinate system is referred to as the world coordinate system. After calibration of the robotic arm 3 and the end effector 9 to the world coordinate system, the LiDAR sensors 17 and 19 are calibrated to correspond their video or distance output from LiDAR sensing to the world coordinate system by a transformation matrix T₁ that converts points in the LiDAR coordinate system into points in the robotic arm world coordinate system.

The patient is then placed on the bed, chair or whatever support is provided, and the patient is sensed and photographed by the LiDAR sensors 17 and 19. The output from the LiDAR sensors 17 and 19 then is used to locate the patient's face in the world coordinate system. That location of the patient's face is used to calibrate the relationship between the hi-def image of the scanned patient data to the location of the patient as located in the LiDAR video and distance scan data as another transformation matrix T₂ that converts points in the hi-def scan mesh model local coordinate system to points of the actual location of the patient's head in the LiDAR system.

These two transformation matrices provide for a rapid way to control the movement of the robotic arm 3 in treatment by the computer system 25 determining the location and orientation required in the treatment plan in the internal hi-def scan coordinate system, then converting that to the location and orientation in the actual LiDAR coordinate system, and then finally converting location and orientation that to the point and orientation to which the robotic arm 3 should move the end effector 9 in the world coordinate system.

The treatment procedure then starts, and the computer system 25 issues electronic instructions to the robot control circuitry 23 that cause the robotic arm 3 to move in a trajectory path in which the end effector 9 is applied to a predetermined series of the treatment segments 53 with the parameters of the treatment procedure that have been defined by the technician. The computer system 25 also sends instructions to the operative part 15 when in place in each treatment segment to effectuate the treatment for that segment, such as, in the preferred embodiment, by causing the microneedles to extend into the skin of the patient at the prescribed depth, and providing the additional RF, heat or light determined in the treatment plan setup.

During the process, the LiDAR continuously monitors and updates the position of the head of the patient and the transformation matrix defining the relationship between the internal hi-def scan mesh model coordinates and the real location of the head coordinates in the LiDAR coordinate system, compensating for any movement of the patient.

Checking processes also are in place during the treatment to address other situations where the patient moves rapidly, or where anomalous forces on the end effector develop, and, responsive to detection of movement or forces above certain predetermined thresholds, the data defining the location of the patient's treatment areas in the world coordinate system is updated, or in appropriate situations robot arm withdraws the end effector from the patient.

### Calibration of the Robotic Arm

The robotic arm 3 and the end effector 9 must first be calibrated so that the computer system 25 has a world coordinate system in which the robotic arm 3 can be correctly commanded to move the end effector 9 to a specific position in the operating theater around the robotic arm 3. This is done by defining a real-world coordinate system used by the computer system 25 and confirming that the electrical directives from the computer system 25 do in fact cause the end effector 9 to be moved to the intended location and orientation specified.

This is accomplished using the calibration system customarily provided with the robotic arm 3 by its manufacturer. In the preferred embodiment of the KUKA robot arm, the robot control is supported by a Robot Operating System (ROS), a meta operating system running on the robot control circuitry 23, which includes a processor and memory allowing it to execute that software. The software includes software packages, including an open-source metapackage for the KUKA robot arm referenced as iiwastack, a proprietary software package called pylbrros used to control the KUKA robot arm in a simpler manner by abstracting the ROS software layer, and an open-source API named rosbridge, which is configured to create interface software tools with the ROS, and is used to set up the interface between the computer system 25 and the robotic arm 3.

The process for calibrating the end effector 9 in the preferred embodiment is as follows:

(i) attaching the end effector 9 to the flange on the end of the robotic arm 3.

(ii) creating or defining a virtual configuration of a model of the end effector 9 in the software running on the computer system 25 including defining specifics of the tool of the end effector 9.

(iii) affixing an object with a sharp point near the robotic arm 3 so the robotic arm 3 can cause the end effector 9 to approach the point from four completely different directions and orientations.

(iv) selecting the tool calibration operation of the software of the SmartPad control for the KUKA robot, for a four-point XYZ tool calibration that relies on the robotic arm outputting its location and orientation.

(v) repeatedly repositioning the end effector tool to a new orientation centered on and touching the sharp point on the object, and direct the software to record the point defined from the feedback of position information output by the robotic arm 3.

The result of this process is that the computer system 25 can rely on the robotic arm's world coordinate system and points defined in it to control precise movement of the robotic arm 3 for the treatment plan, and can generate commands that direct the robotic arm 3 to place the end effector 9 at a location and orientation in the real world defined by the computer system 25.

### Calibration of the LiDAR Stereoscopic Video Sensors to the World

The LiDAR and camera systems/units/sensors 17 and 19 produce both color video and LiDAR data outputs. The video output is a series of video frames of data each comprising a set of pixel data for the resolution of the camera output, where each pixel is associated with respective data that defines RGB color data for that pixel. The LiDAR output is also a field of pixels of a different resolution, where each pixel has data defining the distance determined from the LiDAR sensor to the nearest object in the ray extending through the respective pixel from the sensing point of the LiDAR sensor.

The data output by the LiDAR and camera units 17 and 19 is preferably combined and used to derive a single integrated LiDAR data array comprising data in which each pixel is identified by its row and column in the field of view of the LiDAR sensor, and for each pixel there are six respective data values, the red, green and blue color intensities of the color of the pixel, and the x, y and z coordinate values for the position of the nearest point in the pixel along a ray to the sensor.

In one embodiment, the video calibration is performed using ArUco codes/markers. These are visible codes or markers that are placed in the field of view of the stationary LiDAR sensor 17. The locations of these codes are detected using the video cameras in each of the LiDAR sensors 17 and 19. The end effector 9 on the robotic arm 3 is then manually moved to the center of each ArUco code, and the robotic arm electronics returns its definition of the location of the end effector in the world coordinates, which is recorded. The relative positions of the ArUco center points relative to the LiDAR sensor 17 are determined by averaging the coordinates of the four corners of the ArUco code. It should be noted that other codes or markers can also be utilized to practice the invention.

The result of this is data defining a set of three or more points defined in the LiDAR coordinate system, i.e., x_{L}, y_{L}, z_{L}, and in the robotic arm world coordinate system, i.e., x_{R}, y_{R}, z_{R}. The system herein uses the techniques introduced in S. Umeyama, “Least-squares estimation of transformation parameters between two point patterns,” in IEEE Transactions on Pattern Analysis and Machine Intelligence, vol. 13, no. 4, pp. 376-380, April 1991, doi: 10.1109/34.88573 available at https://ieeexplore.ieee.org/document/88573.

With three or more of these points, as well known to those of skill in the art, a 4×4 transformation matrix T₁ can be derived by using the Umeyama algorithm. This T₁ matrix can be used to transform from the world system coordinates to the LiDAR system coordinates and vice versa.

The transformation matrix T₁ can be used in the computer system 25 to formulate commands to the robotic arm 3 that cause it to interact with the patient at specified real-world points, as will be described below.

For the stationary LiDAR camera unit 17, the transformation matrix T₁ should remain constant unless the LiDAR camera unit 17 is moved accidentally or if there is some alteration in the robotic arm location.

The moving LiDAR camera unit 19 on the robotic arm 3 has a coordinate system that essentially moves with that of the robotic arm 3, so another transformation matrix for its video needs to be updated whenever the robotic arm 3 moves. However, because it moves with the robotic arm 3, the relationship of the coordinate system of the LiDAR camera unit 19 to the world coordinate system is essentially the same as the relationship of the robotic arm position to the world coordinate system.

### Calibration of the Hi-Def Mesh Model to LiDAR Coordinates

The hi-def mesh model of the patient's head is expressed in internal coordinates relative to itself only, and to allow the computer system 25 to calculate the coordinates for instructions for movement of the robotic arm 3 to the real patient's head, the system needs to determine a relationship between the hi-def mesh model coordinates and the coordinates of the location of the patient's head in the real world, which is determined by the LiDAR systems in their own local coordinates.

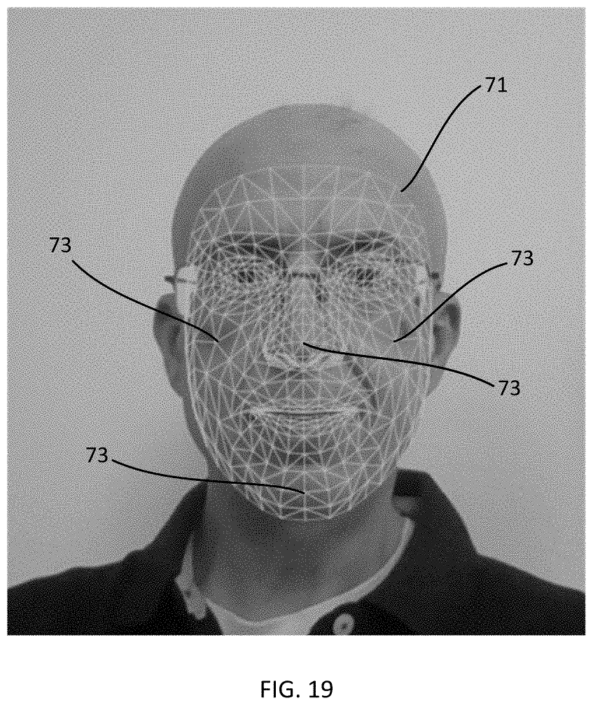

To obtain this relationship and calibration of the two coordinate systems, video from the camera in the LiDAR camera unit 17 is processed by a facial locator program, which in the preferred embodiment is Google FaceMesh, which identifies a human face in a frame or frames of video and can identify from a photograph a number of “landmark” points on a person's face, such as the tip of the nose, the tip of the chin, a cheekbone, etc. FaceMesh does this by creating a mesh in the image as seen in FIG. 19. The wireframe “mask” 71 is aligned by the program with the face in the image, and some of the vertices 73 of the wireframe superimposed on the image are “landmarks” that can be used as identifiable points that are in both the LiDAR image of the patient's head and the hi-def scan mesh model of the patient's head. It should be noted that other facial locator programs can also be utilized to practice the invention.

In some embodiments, a number of landmarks on the patient's head recognizable by FaceMesh are selected by the user or automatically by the computer system 25. The landmark points are selected on the hi-def mesh model and also on the face of the patient in the LiDAR video. At least three points are needed to calibrate the hi-def mesh coordinate system to the LiDAR coordinate system, but at least five should be used, and preferably at least 20 for accuracy.

The landmark points in the LiDAR video image are identified by the pixels where they are located in the image. With the pixel designation for each landmark point in the LiDAR image, the system accesses the data in the integrated LiDAR data array for that pixel and obtains the coordinate of the point in the three dimensional LiDAR coordinate system, i.e., x_{L}, y_{L}, z_{L}. This process of converting from the two-dimensional image pixel location to the three-dimensional depth data coordinates is referred to a “deprojection”.

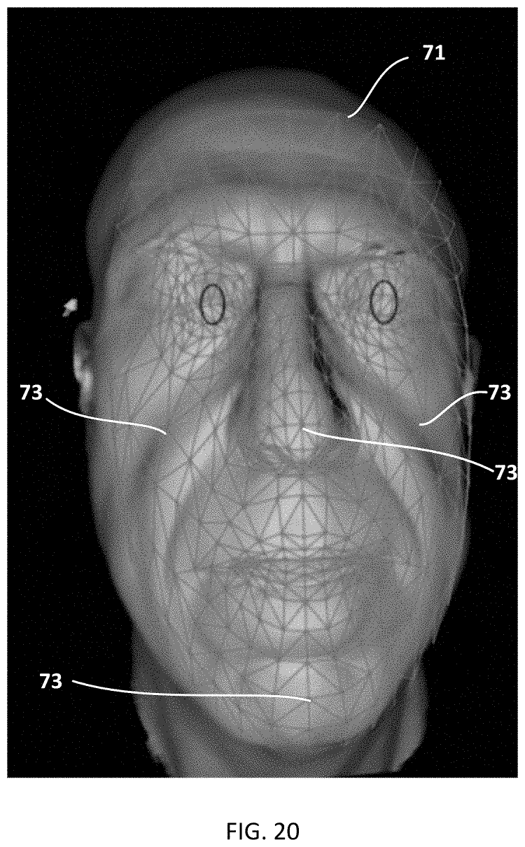

To process the hi-def mesh model, an image is rendered from the hi-def mesh model of the scanned patient's head, and that rendered image is processed by FaceMesh as well, identifying the same landmarks that are identified in the LiDAR image. FIG. 20 shows a rendered hi-def model image processed with FaceMesh so as to apply the FaceMesh “mask” 71 to the image and identify the same landmarks 73 as were indicated in the FaceMesh processing of the LiDAR camera image in FIG. 19. The coordinates in the hi-def mesh model of the scanned patient's head of the same landmark points identified by FaceMesh in the LiDAR imagery are also identified in the calibration process, and the local coordinates of each of those points in the hi-def mesh model, i.e., x_{M}, y_{M}, z_{M}, are compared with the coordinates of the same points in the LiDAR coordinate system, i.e., x_{L}, y_{L}, z_{L}.

From the two coordinate expressions and each of the points, using the method described in the Umeyama article identified above, a transformation matrix T₂ is derived that converts coordinates for a point in the hi-def mask coordinate system, i.e., x_{M}, y_{M}, z_{M}, to the coordinates for that same point in the LiDAR coordinate system, i.e., x_{L}, y_{L}, z_{L}.

$$( \begin{matrix}{x}_{L} \\ {y}_{L} \\ {z}_{L} \\ 1\end{matrix} ) = {T}_{2} ( \begin{matrix}{x}_{M} \\ {y}_{M} \\ {z}_{M} \\ 1\end{matrix} )$$

The FaceMesh program continuously receives the video from the LiDAR sensor 17 and constructs a mask for the face of the patient, including identifying the landmarks used to calculate T₂. Generally, the local hi-def mesh scan model is unchanged as are the specific landmark points, so those coordinates x_{M}, y_{M}, z_{M} do not change over the period of the treatment. However, if there is any movement of the head of the patient, it changes the coordinates of the points in the LiDAR coordinate system, and the transformation matrix T₂ must be re-calculated with the new values of x_{L}, y_{L}, z_{L}, for each landmark point. Accordingly, the FaceMesh program is run constantly to determine if there is any change in the locations of the landmark points on the patient's head.

The transformation matrices T₁ and T₂ are used to convert the coordinates in the hi-def mesh scan model of the patient, which is the coordinate system in which the treatment plan is defined, to coordinates of the robot so that the robotic arm in the real world executes the treatment plan as defined in the hi-def mesh model. This is a two-step conversion, in that first the hi-def mesh coordinates are changed to the LiDAR coordinates, and then those LiDAR coordinates are changed to the robotic arm coordinates for giving real-world instructions to the robot.

### The Treatment Plan and Treatment

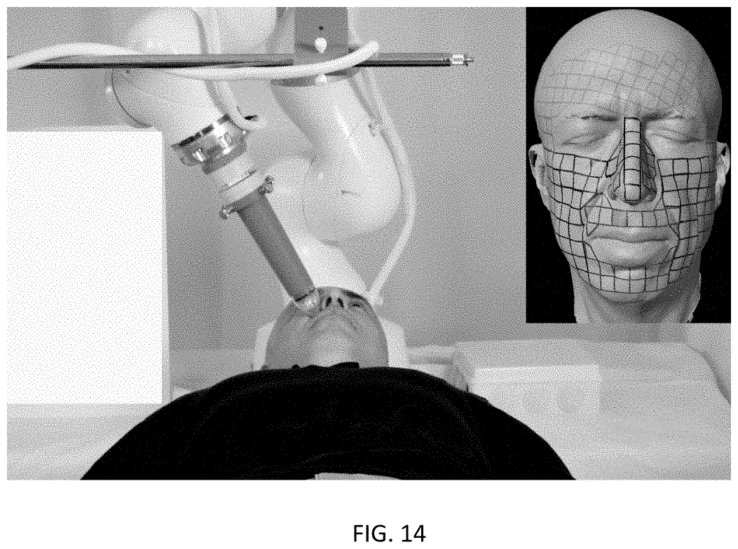

The treatment plan set out in the computer system 25 is followed after the initial setup and calibration is complete. In the treatment plan, the robotic arm 3 is directed to move so as to cause the end effector 9 to engage the skin of the patient in a treatment segment, as seen in FIG. 14. When the treatment of the segment is complete, the robotic arm 3 is withdrawn and moves to the next treatment segment. Essentially, the computer system 25 directs the robotic arm 3 to move to each of the treatment segments of the treatment plan in a prescribed sequence or trajectory, which may be just going through rows of segments in series, or in random order, or may be a more complex processing process. Between individual engagements at treatment segments, the robotic arm 3 withdraws the end effector 9 or tool to a safe distance as it is moved to the next treatment segment.

The treatment segments are defined in the internal hi-def mesh model coordinates. As the treatment procedure proceeds, in whichever order the treatment segments are addressed, the computer system 25 determines the next location for the end effector 9 to be moved to and its orientation. Those points and vectors are determined by the computer system 25 determining the point of application of the end effector 9, which is the center point of the treatment segment, i.e., the center of the square area. The coordinates of that point in x_{M}, y_{M}, z_{M} are multiplied by T₂ to yield the current location in the LiDAR coordinates, x_{L}, y_{L}, z_{L}. Then those point x_{L}, y_{L}, z_{L}, coordinates are multiplied by T₁ to yield the robot world coordinates x_{R}, y_{R}, z_{R} and the robotic arm 3 is directed to move to a place to apply the end effector 9 to that point.

The computer system 25, after identifying the centerpoint, also determines the vector that is normal to the surface of the hi-def mesh at that center point. That orientation vector is then similarly multiplied by T₂ and T₁ to yield the vector that is normal to the centerpoint in the real world robot coordinate system, which is implemented as will be described below.

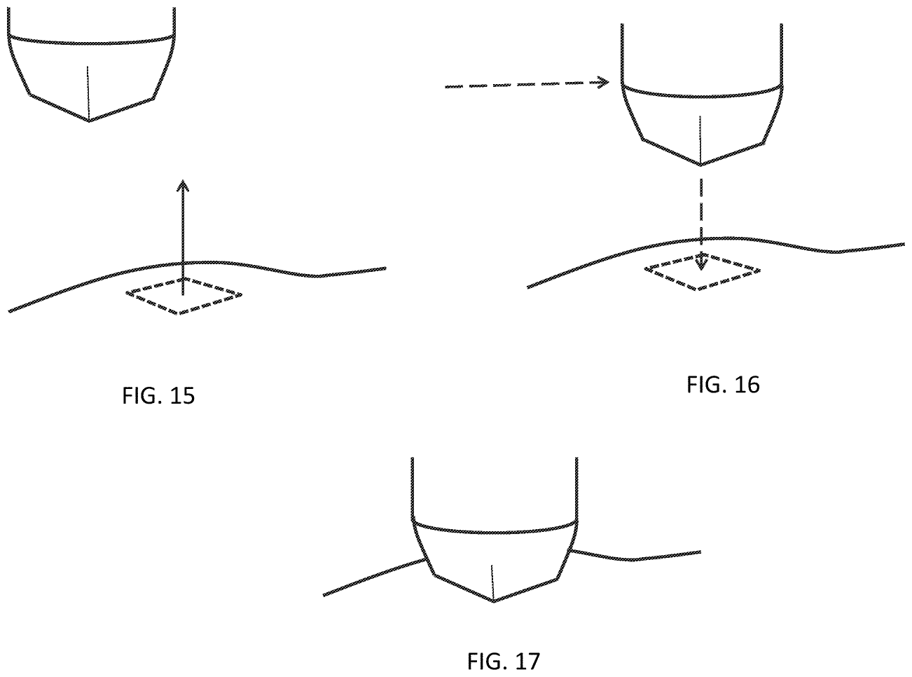

As best seen in FIGS. 15-17, the microneedle tool approaches a treatment segment with the robotic arm 3 being instructed to first proceed to a point and orientation spaced about 15 cm away from the skin of the patient, positioned and oriented to align with the normal to the surface of the treatment segment. Starting from that point, the robotic arm 3 causes the end effector 9 to approach along a path that is normal to the surface of the skin. The tool is advanced along the normal line until it engages the skin of the patient, as seen in FIG. 17. Treatment will proceed if and only if the load sensor 11 determines that the force along the tool z-axis is above a stamp threshold force, but below maximum values. If those requisites are met, the treatment plan begins, and the needles are extended to penetrate the skin of the patient, and to provide any other treatment specified in the treatment plan for that treatment segment, with the duration specified by the user that set up the treatment plan.

The treatment plan continues until completed for all listed treatment segments in the treatment plan.

### Calibration Adjustment for Small Patient Movement

During the treatment procedure, the patient being treated is able to move, and, if the movement detected is below a threshold speed value, the system reacts to the patient's movement by adjusting the transformation matrix T₂ to correct the correspondence between the hi-def head scan and the actual position of the patient's head as detected by the LiDAR cameras.

The video from the LiDAR camera is constantly processed by FaceMesh to determine the position of the patient's face. The LiDAR views of the patient's face may present an issue for the face identification processes of FaceMesh. This may be due to the presence of the end effector occluding view from the camera of part of the patient's face. Generally, the robotic arm will not move the end effector toward the patient where the FaceMesh AI is not returning confident results.

To address this, the imagery is filtered first to remove the pixels that have the color of the end effector. This does not make the occluded landmark points visible, but it does allow for better image processing by FaceMesh because the video more clearly contains facial imagery.

Also, to get a current read on the face position if the end effector is in the way and the location of the face is not clear, the end effector may be elevated out of the way temporarily to allow the LiDAR to sense the location of the patient.

Once FaceMesh locates the face of the patient, the pixels that contain selected landmarks are deprojected, resulting in three-dimensional location data for each. Outlying three-dimensional data points, usually points that have sporadic depth fluctuation due to angled surfaces that do not return uniform depth in the field of view, are discarded.

The locations of each of the landmarks relative to its earlier values are then determined to determine its velocity. The velocities of the landmarks are then totaled and averaged. If the average of the movement velocity of the landmarks on the face is below a threshold velocity, for example, below a centimeter per second, the system determines the transformation matrix T₂ again to confirm to the current location of the patient's face in the LiDAR coordinate system, and the new values of T₂ are used to generate the commands to the robotic arm during the continuing treatment.

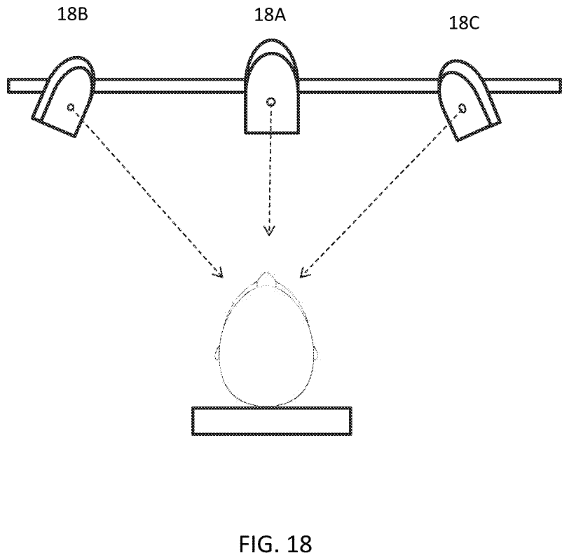

An alternate embodiment of the LiDAR sensors arranged for stereoscopic video is shown in FIG. 18. The sensors and cameras are distributed as follows on a stationary support:

1) a LiDAR sensor 18A is located in the center of the bed above the patient. It faces down at a 50 degree angle and is about 0.5 meters from the tip of the patient's nose.

2) a left-side stereoscopic camera 18B is spaced about 0.25 meters to the left of the LiDAR sensor, angled inward about 40 degrees and downward 50 degrees.

3) a right-side stereoscopic camera 18C is spaced about 0.25 meters to the right of the LiDAR sensor, angled inward about 40 degrees and downward 50 degrees.

The placement of the cameras to the sides creates a superior video imaging of the patient for determining the position of the head of the patient irrespective of the location of the end effector. The FaceMesh AI can rely on one side image with slightly more than half the face and still detect enough landmarks to give a confident read on the location of the patient's head.

### Withdrawal the Robotic Arm

The robotic arm 3 and the end effector 9 also will be withdrawn for safety reasons based on a few system events. Withdrawal means movement in the z-axis of the tool away from the patient. Events that will trigger this are:

(i) if the average velocity of the landmarks exceeds a threshold maximum permitted average velocity, e.g., 1 cm/sec, then it will be assumed that the patient has moved significantly, and the robotic arm will withdraw.

(ii) if the load cell detects a force in the z-axis of the end effector that exceeds a predetermined maximum value, then the robotic arm will withdraw.

(iii) if the load cell detects a force above another threshold in the x- and or y-axis, the robotic arm will withdraw.

(iv) if the operator presses an emergency stop button that is given to the patient, then the robotic arm will withdraw.

After withdrawal, the robotic arm may reposition and return to operation, but only in the cases of large movement or patient emergency button activation will the robotic arm return to operation without some intervention by the user. That intervention is usually simply clicking resume on an alert notification screen on computer system 25.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications and various publications, are cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

What is claimed is:

1. A robotic system for treating a patient, comprising:
a robotic arm with an end effector for treating the patient, wherein the robotic arm is configured to be movable in a space surrounding the patient, and the end effector is configured to be movable individually and co-movable with the robotic arm in said space;
a sensing device for acquiring data associating with coordinates and images of the end effector and the patient; and
a controller in communications with the robotic arm and the sensing device for controlling movements of the robotic arm with the end effector and treatments of the patient with the end effector based on the acquired data and a treatment plan.

2. The robotic system of claim 1, wherein the end effector is supported on a load sensor on the end of the robotic arm so that the end effector is movable by the robotic arm to essentially any location in said space.

3. The robotic system of claim 2, wherein the load sensor is a three-axis load sensor for sensing the forces being applied to the end effector in three orthogonal axes, x, y and z, with the z-axis being the lengthwise axis of the end effector and the x- and y-axes being lateral dimensions perpendicular to that vertical dimension and each other.

4. The robotic system of claim 3, wherein the end effector comprises an operative portion that acts on the patient directly, a tool control circuit for controlling actions of the end effector, and a housing containing the electronic circuitry, wherein a proximal end of a housing is supported on the load sensor, and a distal end of the housing supports the operative portion.

5. The robotic system of claim 4, wherein the end effector is a surgical instrument or a medical instrument.

6. The robotic system of claim 5, wherein the end effector is a scalpel, scissors, an electrocauterizer, a gas plasma treatment tool, and/or a microneedle treatment tool.

7. The robotic system of claim 6, wherein the microneedle treatment tool comprises an array of microneedles configured such that each microneedle is selectively activatable to extend into or retract from the skin of the patient independently.

8. The robotic system of claim 7, wherein the microneedle treatment tool is further configured to apply radio frequency (RF) waveforms, heat, light, and/or drug through the array of microneedles to the skin of the patient for therapy.

9. The robotic system of claim 7, wherein the array of microneedles are supported on a structure in the end effector that selectively extends them out through a planar front face of the end effector and into the skin of the patient, or retracts them back behind the planar front face.

10. The robotic system of claim 9, wherein a force applied to each microneedles to enter the skin of the patient is selected at varied levels.

11. The robotic system of claim 1, wherein the sensing device comprises a first sensing unit and a second sensing unit, wherein the first sensing unit is disposed in said space at a stationary location vertically above the patient and directed at the patient, and the second sensing unit is attached on the end effector, such that during the treatment, the second sensing unit moves with the end effector, while the first sensing remains stationary on its support over the patient.

12. The robotic system of claim 11, wherein each of the first and second sensor units comprises a LiDAR sensor and at least one camera, wherein the LiDAR sensor is configured to determine distances of the LiDAR sensor to surfaces of objects in its field of view, and the at least one camera is configured to acquire stereoscopic images of the objects in its field of view.

13. The robotic system of claim 12, wherein the acquired data by each sensing unit comprises an array of range data, and video data for a field of pixels, wherein the range data for each pixel is an optically derived LiDAR distance value of the distance from the LiDAR sensor to the nearest object met by a ray extending through that pixel from the LiDAR sensor.

14. The robotic system of claim 1, wherein the sensing device comprises
a LiDAR sensor located in a center of the bed above the patient; and
a first stereoscopic camera and a second stereoscopic camera symmetrically located a distance to the left and right of the LiDAR sensor, respectively,
wherein the LiDAR sensor and the first and second stereoscopic cameras are attached on a stationary support.

15. The robotic system of claim 1, wherein the controller is in wired or wireless communications with the robotic arm and the sensing device.

16. The robotic system of claim 15, wherein the controller is configured to receive the acquired data from the sensing device, process the received data to determine the coordinates of the robotic arm and the end effector, instruct the robotic arm to move so as to locate the operative portion of the end effector to a desired location relative to the patient and then the end effector to provide the treatment according to the treatment plan.

17. The robotic system of claim 15, wherein the controller is a computer system, a control console, or a microcontroller unit (MCU).

18. The robotic system of claim 1, wherein the treatment plan defines a series of prescribed treatments in which each treatment comprises treatment segments over the skin of the patient and treatment parameters for that treatment segments.

19. The robotic system of claim 18, wherein the treatment parameters for the microneedle treatment tool of the end effector comprise a heat or temperature setting, a choice of red or blue photodynamic therapy, a depth for the microneedles to be inserted, a duration of the treatment, a number of passes for a particular treatment segment, and/or a frequency of the RF energy to be applied.

20. A method for treating a patient using the robotic system of claim 1, comprising:
scanning a portion of the patient to produce data defining a mesh of surfaces between scanned points that corresponds to the skin surface of the scanned portion of the patient, and deriving rendered views of the portion of the patient from the mesh of surfaces, wherein the portion of the patient includes at least areas for the treatment, the rendered views of the portion of the patient are viewable from any desired angle of view, and the rendered views of the portion of the patient serve as a mesh model coordinate system;
identifying the areas for the treatment in the rendered views of the portion of the patient, and dividing the identified areas for the treatment into treatment segments;
identifying the treatment segments in the rendered views of the portion of the patient, and defining treatment parameters for each treatment segment, wherein the treatment segments and treatment parameters collectively constitute the treatment plan to be executed by the robotic system to provide the treatment to the patient;
calibrating the robotic arm with the end effector to set its location in a three-dimensional (3D) spatial coordinate system that serves as a would coordinate system in which the controller determines instructions for movements of the robotic arm and the end effector for the treatment procedure;
calibrating the first and second sensing units to correspond their video and/or distance output from LiDAR sensing to the world coordinate system by a first transformation matrix that converts points in a LiDAR coordinate system into points in the world coordinate system;
locating the portion of the patient in the world coordinate system from the output of the first and second sensing units, and calibrating the relationship between the image of the scanned data of the portion of the patient to the location of the portion of the patient as located in the LiDAR video and distance scan data as a second transformation matrix that converts points in the mesh model coordinate system to points of the actual location of the portion of the patient in the LiDAR coordinate system; and
performing treatment procedure according to the treatment plan by directing the robotic arm to move in a trajectory path in which the end effector is applied to a predetermined series of the treatment segments with the treatment parameters of the treatment plan, and instructing the operative portion of the end effector when in place in each treatment segment to effectuate the treatment for that segment,
wherein the treatment procedure continues until completed for all the treatment segments in the treatment plan.

21. The method of claim 20, wherein the treatment parameters for the microneedle treatment tool of the end effector comprise a heat or temperature setting, a choice of red or blue photodynamic therapy, a depth for the microneedles to be inserted, a duration of the treatment, a number of passes for a particular treatment segment, and/or a frequency of the RF energy to be applied.

22. The method of claim 21, wherein the treatment proceeds only when the load sensor determines that the force along the tool z-axis is above a stamp threshold force, but below maximum values.

23. The method of claim 22, wherein when the treatment proceeds, the microneedles are extended to penetrate the skin of the patient, and to provide the treatment according to the treatment parameters for that treatment segment.

24. The method of claim 20, wherein as the treatment procedure proceeds, in whichever order the treatment segments are addressed, the controller determines a next location for the end effector to be moved to and its orientation, which is the center point of the treatment segment, and wherein the coordinates of that point in the mesh model coordinate system are multiplied by the second transformation matrix to yield the current location in the LiDAR coordinate system, which in turn are multiplied by the first transformation matrix to yield the world coordinates and the robotic arm is directed to move to a place to apply the end effector to that point.

25. The method of claim 20, further comprising monitoring a movement of the patient during the treatment procedure, wherein the LiDAR continuously monitors and updates the position of the head of the patient and the second transformation matrix defining the relationship between the mesh model coordinates and the real location of the head coordinates in the LiDAR coordinate system, compensating for any movement of the patient.

26. The method of claim 20, further comprising checking processes during the treatment procedure to address situations where the patient moves rapidly, or where anomalous forces on the end effector develop, and, responsive to detection of movement or forces above predetermined thresholds, the data defining the location of the patient's treatment areas in the world coordinate system is updated, or in appropriate situations the robot arm withdraws the end effector from the patient.

27. The method of claim 20, further comprising withdrawing the end effector to a safe distance from the patient, when:
a prescribed treatment at a treatment segment is finished;
an average velocity of landmark points on the portion of the patient exceeds a threshold value of a maximally permitted average velocity;
the load cell detects a force in the z-axis of the end effector that exceeds a predetermined maximum value;
the load cell detects a force above another threshold in the x- and or y-axis; or
the operator presses an emergency stop button that is given to the patient.
Between individual engagements at treatment segments, the robotic arm 3 withdraws the end effector 9 or tool to a safe distance as it is moved to the next treatment segment

28. A computerized device for controlling a robotic system in a medical procedure performed on a patient, comprising:
at least one processor; and
a memory device couple to the at least processor, the memory device containing a set of instructions which, when executed by the at least one processor, cause the robotic system to perform a method for treating the patient, the method comprising:
scanning a portion of the patient to produce data defining a mesh of surfaces between scanned points that corresponds to the skin surface of the scanned portion of the patient, and deriving rendered views of the portion of the patient from the mesh of surfaces, wherein the portion of the patient includes at least areas for the treatment, the rendered views of the portion of the patient are viewable from any desired angle of view, and the rendered views of the portion of the patient serve as a mesh model coordinate system;
identifying the areas for the treatment in the rendered views of the portion of the patient, and dividing the identified areas for the treatment into treatment segments;
identifying the treatment segments in the rendered views of the portion of the patient, and defining treatment parameters for each treatment segment, wherein the treatment segments and treatment parameters collectively constitute the treatment plan to be executed by the robotic system to provide the treatment to the patient;
calibrating the robotic arm with the end effector to set its location in a three-dimensional (3D) spatial coordinate system that serves as a would coordinate system in which the controller determines instructions for movements of the robotic arm and the end effector for the treatment procedure;
calibrating the first and second sensing units to correspond their video and/or distance output from LiDAR sensing to the world coordinate system by a first transformation matrix that converts points in a LiDAR coordinate system into points in the world coordinate system;
locating the portion of the patient in the world coordinate system from the output of the first and second sensing units, and calibrating the relationship between the image of the scanned data of the portion of the patient to the location of the portion of the patient as located in the LiDAR video and distance scan data as a second transformation matrix that converts points in the mesh model coordinate system to points of the actual location of the portion of the patient in the LiDAR coordinate system; and
performing treatment procedure according to the treatment plan by directing the robotic arm to move in a trajectory path in which the end effector is applied to a predetermined series of the treatment segments with the treatment parameters of the treatment plan, and instructing the operative portion of the end effector when in place in each treatment segment to effectuate the treatment for that segment,
wherein the treatment procedure continues until completed for all the treatment segments in the treatment plan.

29. A non-transitory tangible computer-readable medium storing instructions which, when executed by at least one processor, cause a robotic system to perform a method for treating a patient, the method comprising:
scanning a portion of the patient to produce data defining a mesh of surfaces between scanned points that corresponds to the skin surface of the scanned portion of the patient, and deriving rendered views of the portion of the patient from the mesh of surfaces, wherein the portion of the patient includes at least areas for the treatment, the rendered views of the portion of the patient are viewable from any desired angle of view, and the rendered views of the portion of the patient serve as a mesh model coordinate system;
identifying the areas for the treatment in the rendered views of the portion of the patient, and dividing the identified areas for the treatment into treatment segments;
identifying the treatment segments in the rendered views of the portion of the patient, and defining treatment parameters for each treatment segment, wherein the treatment segments and treatment parameters collectively constitute the treatment plan to be executed by the robotic system to provide the treatment to the patient;
calibrating the robotic arm with the end effector to set its location in a three-dimensional (3D) spatial coordinate system that serves as a world coordinate system in which the controller determines instructions for movements of the robotic arm and the end effector for the treatment procedure;
calibrating the first and second sensing units to correspond their video and/or distance output from LiDAR sensing to the world coordinate system by a first transformation matrix that converts points in a LiDAR coordinate system into points in the world coordinate system;
locating the portion of the patient in the world coordinate system from the output of the first and second sensing units, and calibrating the relationship between the image of the scanned data of the portion of the patient to the location of the portion of the patient as located in the LiDAR video and distance scan data as a second transformation matrix that converts points in the mesh model coordinate system to points of the actual location of the portion of the patient in the LiDAR coordinate system; and
performing treatment procedure according to the treatment plan by directing the robotic arm to move in a trajectory path in which the end effector is applied to a predetermined series of the treatment segments with the treatment parameters of the treatment plan, and instructing the operative portion of the end effector when in place in each treatment segment to effectuate the treatment for that segment,
wherein the treatment procedure continues until completed for all the treatment segments in the treatment plan.
